(19) United States
(12) Patent Application Publication
(10) Pub. No.: US 2014/0213910 A1
(43) Pub. Date: Jul. 31, 2014

(54) METHOD AND APPARATUS FOR PERFORMING QUALITATIVE AND QUANTITATIVE ANALYSIS OF BURN EXTENT AND SEVERITY USING SPATIALLY STRUCTURED ILLUMINATION

(71) Applicant: The Regents of the University of California, Oakland, CA (US)

(72) Inventors: Anthony J. Durkin, Irvine, CA (US); Amaan Mazhar, Newport Beach, CA (US); John Quan Minh Nguyen, Pomona, CA (US)

(73) Assignee: The Regents of the University of California, Oakland, CA (US)

(21) Appl. No.: 14/163,568

(22) Filed: Jan. 24, 2014

Related U.S. Application Data
(60) Provisional application No. 61/756,988, filed on Jan. 25, 2013.

(51) Int. Cl. A61B 5/00; G06T 7/00

(52) U.S. Cl. CPC A61B 5/0082; G06T 7/0016; A61B 5/445
USPC 600/477

(57) ABSTRACT

Frequent monitoring of early-stage burns is necessary for deciding optimal treatment and management. Superficial-partial thickness and deep-partial thickness burns, while visually similar, differ dramatically in terms of clinical treatment and are known to progress in severity over time. The disclosed method uses spatial frequency domain imaging (SFDI) far noninvasively mapping quantitative changes in chromophore and optical properties that may be an indicative of burn wound severity. A controlled protocol of graded burn severity is developed and applied to 17 rats. SFDI data is acquired at multiple near-infrared wavelengths over a course of 3 h. Burn severity is verified using hematoxylin and eosin histology. Changes in water concentration (edema), deoxygenated hemoglobin concentration, and optical scattering (tissue denaturation) are statistically significant measures, which are used to differentiate superficial partial-thickness burns from deep-partial thickness burns.

### RELATED APPLICATIONS

The present application is related to U.S. Provisional Patent Application Ser. No. 61/756,988, filed on Jan. 25, 2013, which is incorporated herein by reference and to which priority is claimed pursuant to 35 USC 119.

### GOVERNMENT RIGHTS

This invention was made with government support under RR01192 awarded by National Institutes of Health and USAF/AFSOR Grant, No. FA9550-04-0101. The government has certain rights in the invention.

### BACKGROUND

1. Field of the Technology

The disclosure relates to the field of methods based on spatial frequency domain imaging for quantitatively assessing burn severity using the recovered absorption and reduced scattering properties in tissue.

2. Description of the Prior Art

Thermal injuries can be caused by exposure to a wide variety of sources including heat, electricity, radiation, chemicals, and friction. According to the American Burn Association, approximately 500,000 people seek treatment for burn injuries every year. Of that population, about 45,000 have burn injuries requiring medical treatment with about 3500 cases resulting in death.

Skin burns are normally characterized by depth of injury. Starting with the least serious, superficial burns involve injury to the top epidermis layer. These often have a reddish nonblistering appearance (e.g., sunburns) due to increased blood flow to the dermis. They are often hypersensitive to touch and heal in less than two weeks via re-epithelialization. On the other end of the spectrum are full thickness burns, which extend beyond the epidermal and dermal layers of the skin into the subcutaneous layer. These burns often appear leathery, firm, and depressed and are insensitive to pinpricks due to complete destruction of the dermis including nerves and vasculature. Full thickness burns require full excision and grafting as a treatment. Both superficial and full thickness burns are relatively easy to diagnose based on clinical observation.

In between these two extremes are superficial-partial thickness and deep-partial thickness burns in which damage extends to a fraction of the dermal layer. Superficial-partial thickness burns extend to only the upper layers of the papillary dermis, and depending on the extent of damage and remaining vasculature, these injuries may naturally heal in 2 to 3 weeks with minimal to no scarring. Deep-partial thickness burns extend in depth to the reticular dermis and can often be found mixed with portions of noncharred full-thickness burns. These burns often require excision and grafting for optimal treatment. Both categories of partial thickness burns often have a mottled pink and white appearance that can bleach with pressure, and are less sensitive to pinpricks than normal. Partial thickness burns are therefore challenging to identify based on clinical impression. Depending on the clinician's experience, visual assessment has been shown to have a field accuracy of about 50% to 76%. Overestimation of burn severity results in unnecessarily invasive surgical treatment and prolonged hospitalization, whereas underestimation results in treatment delays, extended hospital stays, and increased chances of contracture and hypertrophic scar formation.

Further complicating the situation, burns also undergo dynamic burn wound conversions during the early 48-hr period in which superficial-partial thickness burns have been observed to progress into deep-partial thickness or full thickness burns. The conversion process is not fully understood, but it is generally agreed that continuous monitoring of early-stage burns is necessary for deciding optimal treatment and management. Tissue punch biopsy and histological analysis, despite being the long-standing gold standard for determining burn depth, are far from ideal. Apart from being an invasive and time consuming process that requires the presence of an experienced pathologist, it is also vulnerable to sampling errors due to burn area heterogeneity.

Currently, there are a number of optical imaging technologies in development that may be used to estimate burn depth noninvasively. Technologies such as near-infrared spectroscopy (NIRS) and hyperspectral imaging provide information about burned tissue health by measuring clinically relevant chromophores such as blood oxygen saturation and water concentration. Laser Doppler imaging (LDI) correlates tissue blood flow with burn seventies, whereas optical coherence tomography (OCT) correlates structural collagen denaturation with burn depth. However, these technologies are not without their limitations. LDI, NIRS, and OCT are generally point-based instruments that require patients to remain completely still during potentially long acquisition times in order to acquire full area scans, whereas hyperspectral imaging techniques often require assumptions about tissue optical scattering properties that may not be valid in damaged tissue.

As of now, perfusion monitoring LDI is the only Food and Drug Administration-approved commercial technology for monitoring burns. Despite an often reported accuracy of up to 97% when utilized at least 48 hr post-burn, LDI is markedly less accurate (54% to 80%) when utilized within the first 24 hr of injury due to the effects of reactive vasoconstriction. Additionally, LDI is sensitive to errors caused by fluctuations in the ambient temperature, the patient's emotional state, and the shifts in blood pooling resulting from the patient's position during imaging.

Spatial frequency domain imaging (SFDI) is a noncontact wide field optical imaging technology currently being developed at the Beckman Laser Institute and Medical Clinic in Irvine, Calif. By combining periodic spatially modulated illumination with a camera-based imaging system, SFDI is capable of quantifying wide-field subsurface optical properties which can then be utilized to quantify chromophore concentrations for in vivo tissue.

During imaging, spatially modulated illuminations are projected onto the region of interest over a range of wavelengths. Diffusely reflected light is recorded using a charge-coupled diode (CCD) camera and then demodulated in order to extract the diffuse reflectance at each wavelength and spatial frequency, which can then be further reduced into absorption (μₐ) and reduced scattering (μ′ₛ) coefficients by fitting to a known forward model. Previous validation studies originating from our group using tissue-simulating phantoms have reported an accuracy of approximately 6% and 3% in absorption and reduced scattering parameters respectively. In terms of sensitivity, the same study has also shown that a 1% change in absorption or scattering produces at most an approximate 0.3% change in diffuse reflectance. Biological chromophores are fit to Beer's law using a least-squares fit. With the ability to interrogate skin depths of about 1 to 5 mm, SFDI is able to measure spatially resolved concentrations of clinically relevant chromophores including oxyhemoglobin, deoxyhemoglobin, lipid, water, and tissue oxygen saturation. In addition, SFDI is able to measure quantitative wide-field reduced scattering coefficients at each wavelength. Within the context of burns, the ability to measure the changes in scattering has the potential to confer information related to the changes in structure resulting from denaturation.

In U.S. Pat. No. 6,958,815, incorporated herein by reference, we presented a disclosure involving wide field, broadband, spatially modulated illumination of turbid media. This approach has potential for simultaneous surface and subsurface mapping of media structure, function and composition. This method can be applied with no contact to the medium over a large area, and could be used in a variety of applications that require wide-field image characterization. The approach described in U.S. Pat. No. 6,958,815 is further refined and a fluorescence imaging capability described in U.S. Pat. No. 7,729,750, incorporated herein by reference. Use of wide field, broadband, spatially modulated illumination for wound assessment is disclosed in Cuccia et al., “Method For Performing Qualitative And Quantitative Analysis Of Wounds Using Spatially Structured Illumination”, US Pub. 2010/0210931 (2010), incorporated herein by reference.

### BRIEF SUMMARY

The development of noninvasive technology for evaluation of tissue status is essential for optimizing therapeutic treatments of burn wounds. Thermal injuries are clinically classified according to the depth of the injury as superficial, partial thickness or full thickness. Superficial burns are mild burns whereby the tissue is capable of regenerating the epidermis. Partial thickness injuries destroy a portion of the dermal layer and re-epithelialization can occur if there is sufficient dermis with an adequate vasculature. Full thickness injuries involve destruction of the dermal layer and the reduced blood supply will result in ischemia and necrosis.

Superficial burns are typically treated with dressings and covered, whereas full thickness burns are excised and closed or grafted. Both the superficial and full-thickness burns are readily diagnosed and the most difficult to assess are the partial thickness burns. Overestimation of the burn depth in partial thickness burns would result with invasive excisional treatment and underestimation would delay appropriate treatment and potentially lead to infection.

In addition, burns undergo dynamic changes at the early stages that must be monitored continually for proper burn management. The ultimate decision to graft or to allow re-epithelialization of the wound is left up to the surgeon's discretion after visual Inspection, which can happen a week or so after the Injury.

Spatial frequency domain imaging (SFDI) shows great promise for quantitative imaging of optical properties of superficial (1-5 mm depth) tissues in vivo [e.g. Pixel-by-pixel demodulation and diffusion-model fitting of spatial frequency data is performed to extract the local absorption and reduced scattering optical coefficients. When combined with multispectral imaging, absorption spectra at each pixel can be separately analyzed to yield spatial maps of local oxy-hemoglobin (ct0₂Hb), deoxy hemoglobin concentration (ctHHb), and water concentration (ctH₂0). Total hemoglobin (ctHbT) and oxygen saturation (stO˜ maps can then be calculated as ctHbT=ctHHb+ct0₂Hb and St0₂=100*ct0₂Hb/ctHbT, respectively. We have previously disclosed that these parameters can be used to track wound progression in burns and ulcers. However, it is to be expressly understood that the invention can be practiced using not only diffusion-model fitting of spatial frequency data, but there are many different ways to analyze light propagation in tissue. Diffusion approximation is but one way, and there are other approaches to analyzing light transport including generating forward solutions using Monte Carlo simulation to precalculate a look-up table of spatial frequency dependent optical properties and then upon making SFDI measurements, and using that look up table to assign optical properties. Any methodology now known or later devised can be employed in the claimed invention.

Additionally, the reduced scattering parameters can be recovered at discrete wavelengths on a pixel by pixel basis using SFDI. These wavelength-dependent scattering parameters can then be fit to a power law (μₛ′=A*λ^{-b}) at each pixel. It has been shown A is proportional to the number of scattering particles in the turbid medium and b is proportional the average size of the scattering particles in the medium. Mie scattering occurs when the size of scattering structure is larger than input wavelength and means the b slope is much larger. Rayleigh scattering occurs when sizes of the scattering structures are much smaller than the wavelength and the b value is higher. In skin, the relatively large size of collagen fibrils (about 2-3 μm diameter) give scattering slope skin a particular scattering shape that approaches Mie scattering in the near infrared and Rayleigh in the visible. A plot using data is shown below (FIG. 3).

The baseline values can vary between skin types and representative values are used below. However, when the tissue is burned, these fibrils are denatured due to thermal interactions and the average scattering size gets smaller (b will increase) and the number of scattering parameters will increase (A gets larger). Thus, SFDI can measure the wavelength dependent A and b parameters in a two dimensional map and provide an indication of the thermal damage to the collagen fibril network. The changes in A and b are caused by denaturation of collagen fibrils and can then be correlated to burn severity and extent as shown in animal models previously.

Finally, it should be said that in addition to providing a means for assessing burn wound severity, this method is also likely to have utility as a means to assess wound healing in general. Wound tissue is dynamic and the process of healing involves creation of granulation tissue, accompanied by wound contracture and replacement of granulation tissue by scar tissue, which in many cases eventually normalized. We expect that the wound healing process can be characterized by changes in scattering that reflect the different phases of the healing process. This may be of particular interest with respect to chronic wounds such as those that occur in diabetic subjects, and the development of effective management strategies and therapeutic compounds fur these wounds.

The illustrated embodiment of the invention includes a method using spatial frequency domain imaging (SFDI) for quantitative imaging of optical properties of superficial (1-5 mm depth) tissues in vivo comprising the steps of pixel-by-pixel demodulation and diffusion-model fitting of spatial frequency data performed with multispectral imaging to extract the local absorption and reduced scattering optical coefficients, and separately analyzing absorption spectra at each pixel to yield spatial maps of local oxy-hemoglobin (ct0₂Hb), deoxy hemoglobin concentration (ctHHb), and water concentration (ctH₂0).

The total hemoglobin (ctHbT) and oxygen saturation (stO) maps are calculated as ctHbT=ctHHb+ct0₂Hb and St0₂=100*ct0₂HbT, respectively.

The total hemoglobin (ctHbT) and oxygen saturation (stO) are used to track wound progression in burns and ulcers.

The method further includes the steps of recovering the reduced scattering parameters at discrete wavelengths on a pixel by pixel basis using SFDI and fitting the reduced scattering parameters to a power law (μₛ′=A*λ^{-b}) at each pixel, where A is proportional to the number of scattering particles in the turbid medium and b is proportional the average size of the scattering particles in the medium.

In summary, the illustrated embodiments of the method use spatial frequency domain imaging (SFDI) for quantitative noninvasive, noncontact assessment of severity of burn injury to tissue in vivo. The includes the steps of performing wide-field quantitative mapping of tissue optical properties including pixel-by-pixel demodulation and diffusion-model fitting of spatial frequency data performed with multispectral imaging to extract the local absorption and reduced scattering optical coefficients. Quantitative analysis of burn injury of tissue is performed by separately analyzing absorption spectra at each pixel to yield a spatial map of blood oxygenation, water concentration, optical scattering changes or a combination thereof. The severity of the burn injury to tissue is classified according to the depth of the injury.

The method further includes the step of tracking wound progression of burn injury in tissue in vivo over tame.

The step of performing quantitative analysis of burn injury of tissue by separately analyzing absorption spectra at each pixel to yield the spatial map of blood oxygenation includes performing quantitative analysis of burn injury of tissue by separately analyzing absorption spectra at each pixel to the yield spatial map of local oxy-hemoglobin (ct0₂Hb), deoxy hemoglobin concentration (ctHHb), and water concentration (ctH₂0).

The step of performing quantitative analysis of burn injury of tissue by separately analyzing absorption spectra at each pixel to the yield spatial map of local oxy-hemoglobin (ct0₂Hb), deoxy hemoglobin concentration (ctHHb), and water concentration (ctH₂0) further includes the step of performing quantitative analysis of burn injury of tissue by separately analyzing absorption spectra at each pixel to yield the spatial map of total hemoglobin (ctHbT) and oxygen saturation (St0₂) calculated as ctHbT=ctHHb+ct0₂Hb and St0₂=100*ct0₂Hb/ctHbT, respectively.

The method further includes the step of tracking wound progression in burns using total hemoglobin (ctHbT) and oxygen saturation (St0₂).

The step of performing quantitative analysis of burn injury of tissue by separately analyzing absorption spectra at each pixel to the yield spatial map of optical scattering changes includes recovering the reduced scattering parameters at discrete wavelengths on a pixel by pixel basis using SFDI and fitting the reduced scattering parameters to a power law (μₛ′=A*λ^{-b}) at each pixel, where A is a parameter proportional to the number of scattering particles in the turbid medium and b is a parameter proportional the average size of the scattering particles in the tissue.

The method further includes measuring the wavelength dependent A and b parameters in a two dimensional spatial map to provide an indication of the thermal damage to the collagen fibril network, where the changes in A and b are caused by denaturation of collagen fibrils and can then be correlated to burn severity and extent.

The method further includes the step of assessing wound healing as characterized by changes in scattering that reflect the different phases of a healing process.

The method further includes the step of selecting regions of the spatial map to compare the A and b parameters for all selected regions, where the A value and b are larger for selected regions with a more severe burn.

The severity of the burn is assessed to be related to the amount of change in the A and b parameters.

The change in A and b is due to a change in collagen fibril structures. A is proportional to the number of scattering particles in the tissue and b is proportional the average size of the scattering particles in the tissue. When the tissue is burned, the collagen fibrils are denatured due to thermal interactions and the average scattering size gets smaller so that b increases, and the number of scattering particles will increase so that A increases. The use of SFDI measures of the A and b parameters when displayed in a two dimensional map provide an indication of the thermal damage to the collagen fibril network with the changes in A and b being caused by denaturation of collagen fibrils correlated to burn severity and extent.

The step of performing quantitative analysis of burn injury of tissue by separately analyzing absorption spectra at each pixel to yield a spatial map of optical scattering changes further includes using optical absorption data to map tissue chromophores for determining burn treatment.

The step of performing quantitative analysis of burn injury of tissue by separately analyzing absorption spectra at each pixel to yield a spatial map of water concentration to predict buildup of edema and ischemia progression.

The method further includes the step of predicting burn injury healing progression based on SFDI-related parameters.

The method further includes using multimodal imaging using SFDI and perfusion-based imaging to characterize and analyze vascular changes occurring within a burn wound.

The scope of the invention also includes a method of using spa frequency domain imaging (SFDI) for quantitative noninvasive, noncontact assessment of severity of burn injury to tissue in vivo including the steps of performing wide-field quantitative mapping of tissue optical properties; separately analyzing optical properties to generate a spatial map of blood oxygenation, water concentration, optical scattering changes or a combination thereof; and determining the severity of the burn injury to tissue according to the spatial map of blood oxygenation, water concentration, optical scattering changes or a combination thereof.

The method further includes the step of tracking progression of burn injury in tissue in vivo over time to determine treatment of the burn injury.

The method further includes treating the burn injury according to the spatial map of burn injury in terms of blood oxygenation, water concentration, optical scattering changes or a combination thereof.

The step of separately analyzing optical properties to general spatial map of blood oxygenation, water concentration, optical scattering changes or a combination thereof includes separately analyzing absorption spectra at each pixel to generate the spatial map of local oxy-hemoglobin (ct0₂Hb), deoxy hemoglobin concentration (ctHHb), and water concentration (ctH₂0).

The step of separately analyzing optical properties to generate a spatial map of blood oxygenation, water concentration, optical scattering changes or a combination thereof include recovering the reduced scattering parameters at discrete wavelengths on a pixel by pixel basis using SFDI and fitting the reduced scattering parameters to a power law (μₛ′=A*λ^{-b}) at each pixel, where A is a parameter proportional to the number of scattering particles in the turbid medium and b is a parameter proportional the average size of the scattering particles in the tissue.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

The specification contains at least one drawing executed in color. Copies of this patent or patent application publication with color drawing(s) will be provided by the Office upon request and payment of the necessary fee.

FIG. 1a is a photograph of the brass comb used to create burns.

FIG. 1b is a photograph of a burn wound immediately after injury by applying the heated brass comb of FIG. 1a to the rat's dorsal skin.

FIG. 2 is a diagram of the SFDI system used in the illustrated embodiment to assess burn severity.

FIG. 3 is a microphotograph of H&E stained cross-sections for each burn severity. Dotted lines represent estimated depth of burn.

FIGS. 4a-4d are graphs which show the percent changes of oxygenated hemoglobin concentration in FIG. 4a, deoxygenated hemoglobin concentration in FIG. 4b, total hemoglobin concentration in FIG. 4c, and oxygen saturation in FIG. 4d over the 3-h imaging period for both partial thickness burn populations. Regions of interests were selected directly over burn wounds. Time points with significant differences are marked with an asterisk.

FIG. 5 is a SFDI oxygen saturation maps for two partially burned rats measured at normal baseline, 10 min after injury, and 3 h after injury. Oxygen saturation) is at the far right.

FIG. 6 show SFDI deoxygenated hemoglobin concentration maps for two partially burned rats measured at normal baseline, 10 min after injury, and 3 h after injury. Deoxygenated hemoglobin concentration (μM) is at the far right.

FIG. 7 is a graph of the time plot of average ΔH₂O over the 3-h imaging period for both partial thickness burn populations. Regions of interests were selected directly over burn wounds. Time points with significant differences are marked with an asterisk.

FIG. 8 shows SFDI water concentration maps for two partially burned rats measured at normal baseline, 10 min after injury, and 3 h after injury. Water fraction scale (%) is at the far right.

FIG. 9 is a graph of time plot of average Δb over the 3-h imaging period for both partial thickness burn populations. Regions of interests were selected directly over burn wounds. Time points with significant differences are marked with an asterisk.

FIG. 10 shows SFDI scattering b parameter maps for two partially burned rats measured at normal baseline, 10 min after injury, and 3 h after injury. Scattering b parameter scale is at the far right.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

In the illustrated embodiments we use SFDI in order to quantitatively evaluate burn wound seventies in a rat model. The objective is to map quantitative changes in spatially resolved tissue oxygenation, water concentration, and reduced scattering that may be indicative of burn wound severity and relate these changes to burn severity as reported by hematoxylin and eosin (H&E) histology.

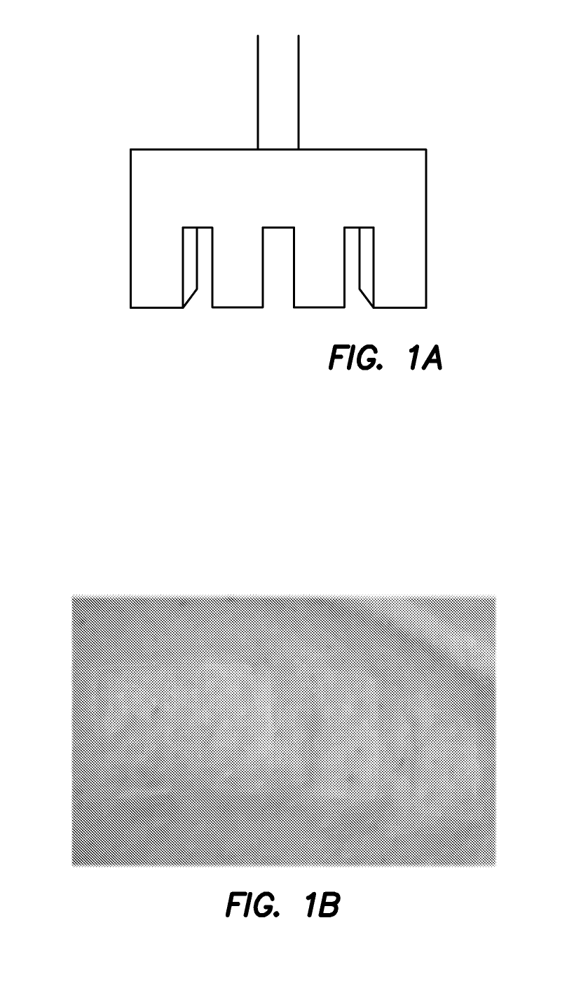

Seventeen male Sprague-Dawley rats, weighing 350 to 600 g, used in the study of the illustrated embodiment of the method. Burn injuries were created using a previously established heated “brass comb” shown in FIG. 1a. The custom-made comb weighed 313 g and consisted of four notches measuring 1×2 cm² separated by 0.5 cm gaps. On the day before experiments, each rat was shaved along the lateral dorsal region of the body using electric clippers and depilated with Nair (Church and Dwight, Princeton, N.J.). During experiments, the rats were anesthetized using an intraperitoneal injected mixture of ketamine hydrochloride and xylazine with additional boosters administered as necessary. The brass comb was heated to 100° C. via immersion in a boiling water bath and applied onto the shaved lateral dorsal region without additional pressure (gravity only) for 2 to 15 s in order to create burns of graded severity, ranging from superficial-partial thickness to full thickness. After imaging the rats every 10 to 20 min for 3 h post-burn, the rats were euthanized using pentobarbital, and the burn region was excised into 10% buffered formalin where they were fixed for 24 h before being prepared for histology. In each case, burn severity was verified using standard H&E staining and optical microscopy (Olympus BH2, Tokyo, Japan).

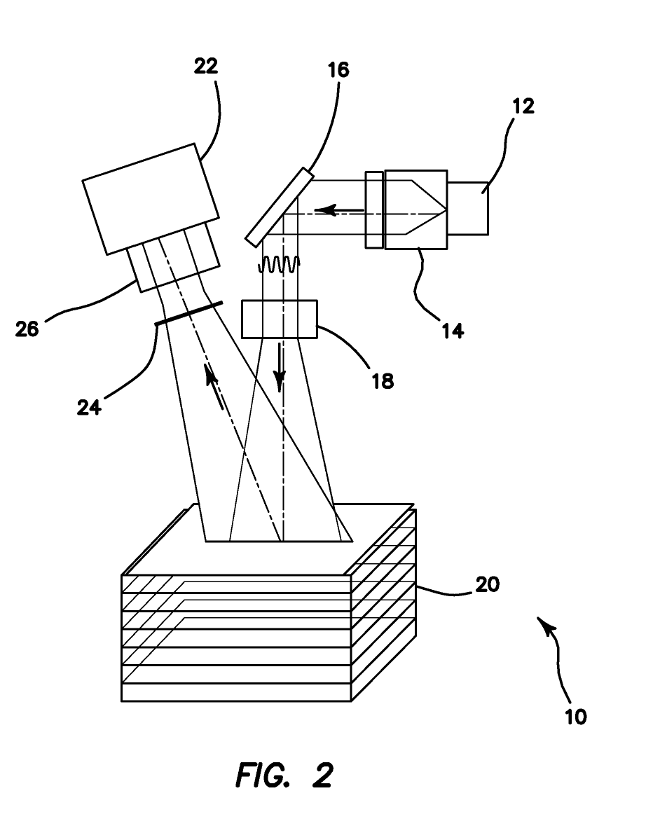

The SFDI instrument 10 used in this study comprised a 250-W tungsten lamp light source 12 (Newport Oriel, Stratford, CT) focused by optics or condenser 14 and used to illuminate spatially modulated projections created by a digital micromirror device (DMD) 16 (Texas Instruments, Dallas, Tex.) at an illumination angle of 0 deg. A diagram illustrating the instrumentation 10 can be seen in FIG. 2; however, for the sake of visual clarity, the actual illumination angle is not accurately depicted. Diffusely reflected light from tissue or specimen 20 was captured using a multispectral CCD camera 22 (Nuance, Cri, Inc., Woburn, Mass.) including a liquid-crystal tunable filter 26 for wavelength selection and a pair of linear cross polarizers 24 to reject specular diffuse reflectance. Images were saved as binary files for post-acquisition processing via MATLAB (MathWork Natick, Mass.). Other SFDI systems could be employed according to user preference and the essential elements of instrumentation 10 have been illustrated only for example.

For the purpose of this study, a 65×86 mm² field-of-view was imaged over the course of 3 h at two spatial frequencies: 0 and 0.1 mm⁻¹. Seventeen spectral wavelengths between 650 and 970 nm were acquired in 20 nm intervals, and the diffuse reflectance images were calibrated for system 10 response using tissue-simulating phantoms with known optical properties (μₐ=0.0176 mm⁻¹ and μ′s=1.024 mm⁻¹ at 650 nm). Effects related to variations in height and curvature were ameliorated using a conventional three dimensional profile intensity correction as described by Gioux at al. “Three-dimensional surface profile intensity correction for spatially modulated imaging,” J. Biomed. Opt. 14(3), 034045 (2009). Pixel-by-pixel optical property values were calculated using a two-frequency look-up-table approach based on Monte-Carlo forward predictions.

Chromophore concentrations were calculated from the absorption spectrum according to Beers law. In addition, reduced scattering (μ′ₛ) was fit to a model based on infrared Mie theory approximation, μ′ₛ=Aλ^{-b}, where λ is the imaging wavelength and A and b are free variables determined by a least squares fit. The scattering b parameter, in particular, was analyzed in this study as it is a possible indicator of scattering moiety size changes related to tissue denaturation.

In order to determine if there was a statistically significant difference in each SFDI derived parameter at each time point, a Welch's West was used to compare the relative changes between superficial-partial thickness and deep-partial thickness burns. We have chosen to focus on these two burn types in particular as the differentiation between these two burn types is clinically difficult, as discussed above. A p-value of less than 0.05 was considered significant for this study.

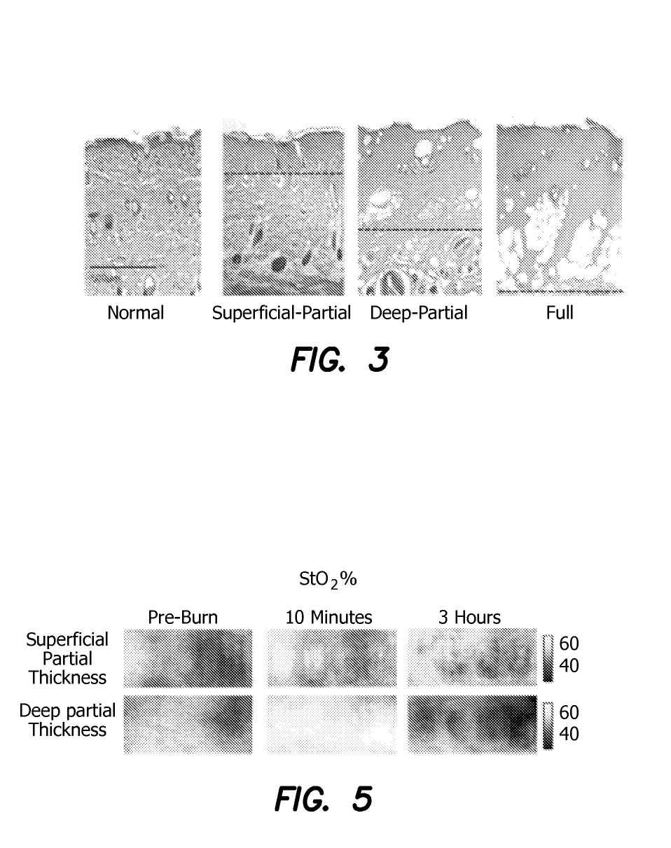

The development of noninvasive technology for evaluation of tissue status is essential for optimizing therapeutic treatments of burn wounds. Thermal injuries are clinically classified according to the depth of the injury as superficial, partial thickness or full thickness. Superficial burns are mild burns whereby the tissue is capable of regenerating the epidermis. Partial thickness injuries destroy a portion of the dermal layer and re-epithelialization can occur if there is sufficient dermis with an adequate vasculature. Full thickness injuries involve destruction of the dermal layer and the reduced blood supply will result in ischemia and necrosis. Burn severities for each sample were verified, post-experiment, via H&E staining and optical microscopy. Depth of burn was determined, as suggested by the literature and in concurrence with a specialized clinician, by examining for viable adnexal structures (such as hair follicles and sweat gland and by examining for the appearance of glass-like collagen hyalinization. Examples of H&E stained cross-sections for the burn types normal, superficial-partial, deep-partial and full as seen in FIG. 3. In developing our rat burn model, we were able to induce 20 superficial-partial thickness burns, 34 deep-partial thickness burns, and 8 full thickness burns.

After examining all of the chromophore and optical property data that resulted from the study, SFDI data analysis was concentrated on three parameters that may be useful for differentiating partial thickness burns: blood oxygenation, water concentration, and optical scattering changes. Time plots with standard deviations were generated from the averages of each burn severity population. To illustrate SFDI's wide-field imaging capabilities, maps of oxygen saturation, deoxygenated hemoglobin concentration, water concentration, and scattering b parameter for two partially burned rats are presented.

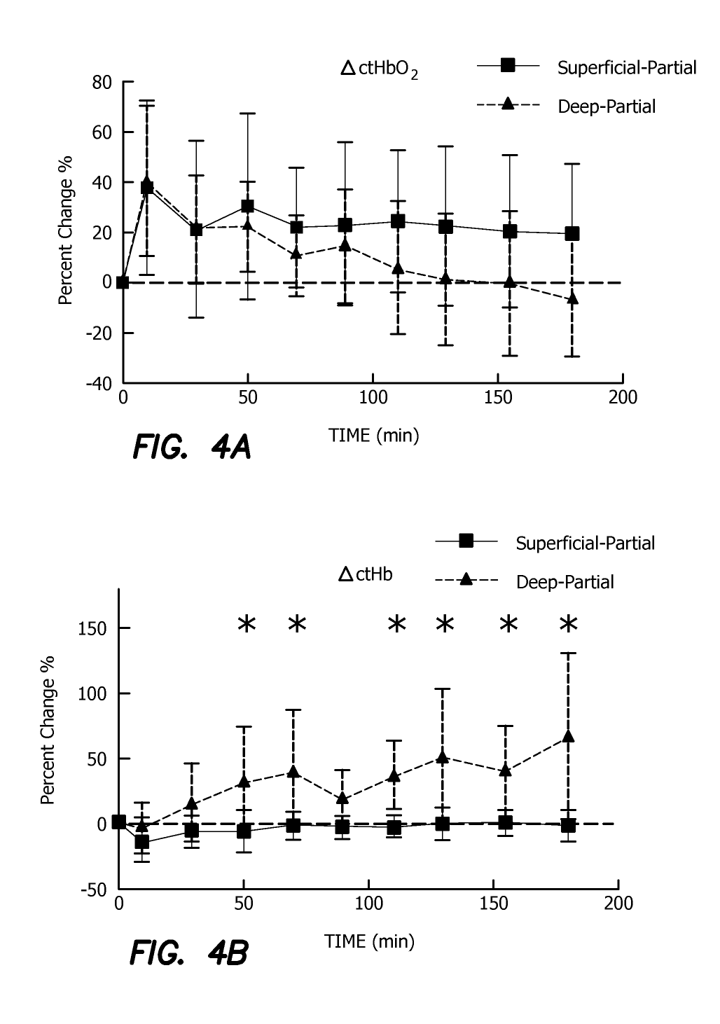

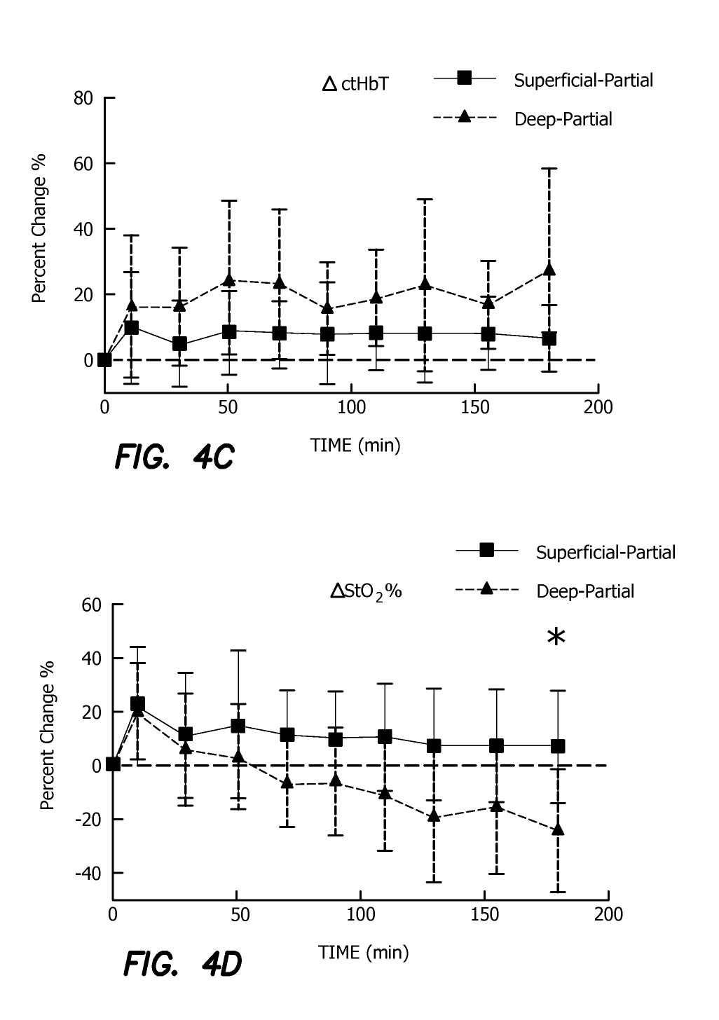

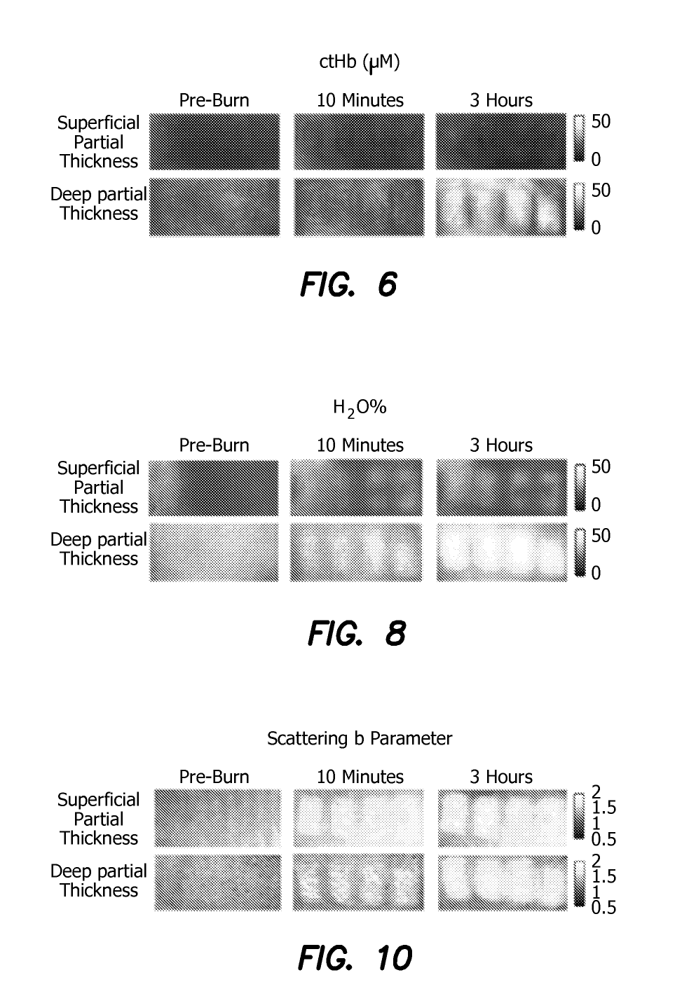

Relative changes in oxygenated hemoglobin concentration (ctHbO₂), deoxygenated hemoglobin concentration (ctHb), total hemoglobin concentration (ctHbT), and blood oxygen saturation (StO₂%) are shown in FIG. 4. SFDI-generated StO₂% maps for two partially burned rats can be seen in FIG. 5 and SFDI generated ctHb maps for the same two rats can be seen in FIG. 6. While no significant differences were observed in the relative changes for ctHbO₂ and ctHbT, significant differences in the relative changes of ctHb were observed starting at 50 min postburn (p=0.035). Differences in StO₂% were not statistically significant until 3 h post-burn (p=0.022).

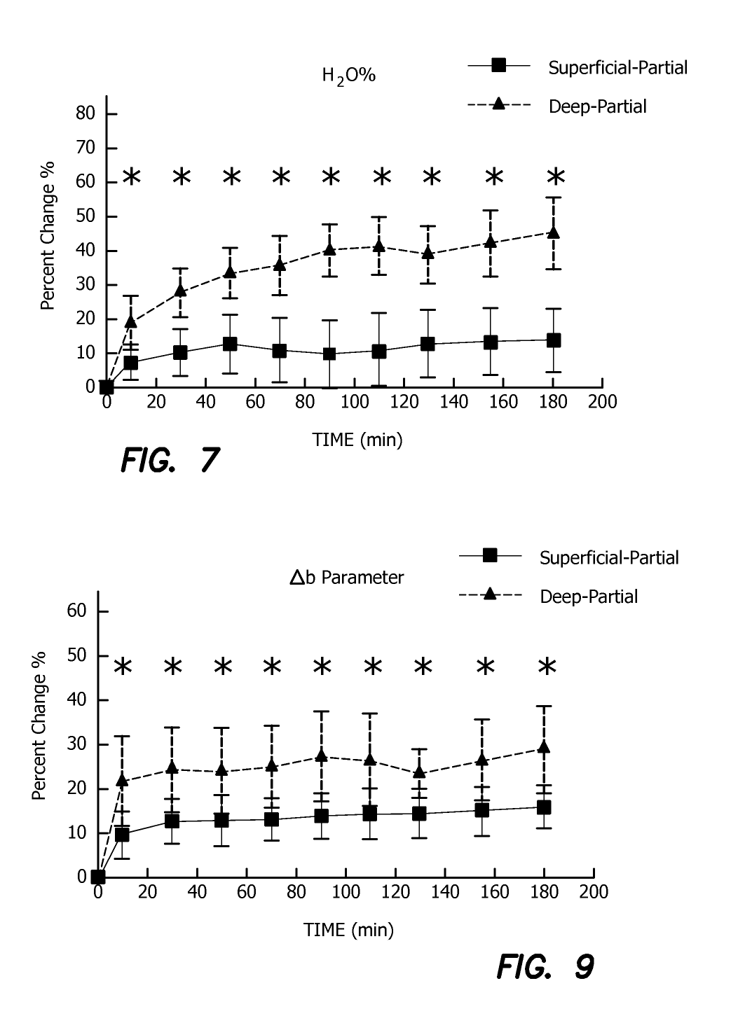

Relative changes in water concentration (H₂O %) are depicted in FIG. 7, and SFDI-generated H₂O % maps for two partially burned rats can be seen in FIG. 8. Statistically significant differences in H₂O % were observed starting at 10 min postburn (p=0.009).

Relative changes in scattering b parameter can be seen in FIG. 9, and SFDI-generated b maps for two partially burned rats can be seen in FIG. 10. Significant differences in scattering b parameter were observed starting at 10 min post-burn (p=0.024).

For the first time, what our laboratory has developed is a model of graded burn severity for SFDI. As discussed above, SFDI can be used to monitor chromophore concentrations and optical properties over a wide field-of-view. During the 3-h post-burn period, we were able to observe dynamic changes in blood oxygenation, water concentration, and optical scattering that have potential for aiding the process of assessing burn severity.

Tissue oxygenation maps may be an indicator of vascular damage and patency. As seen in FIGS. 4a-6, we were able to observe how tissue blood oxygenation varied depending on burn severity. Within the first 10 min after burning, an approximately 40% increase in ctHbO₂ was observed for both partial thickness burns, possibly due to inflammatory vasodilation and increased oxygenated blood flow into the injured regions. During the remainder of the 3-h post-burn imaging period, ctHbO₂ for deep-partial thickness burns gradually decreased to 10% below baseline, whereas ctHbO₂ for superficial-partial thickness burns remained about 25% above baseline. At the same time, ctHb for deep-partial thickness burns gradually increased to about 50% above baseline while ctHb for superficial-partial thickness burns remained close to baseline. This gradually significant increase in ctHb over time combined with the complimentary ctHbO₂ decrease in the deep partial-thickness burn may be indicative of capillary blood stasis due to deep dermis thrombosis and ischemia resulting from the burn. The same trend is not observed in the superficial-partial thickness burns, suggesting that much of the underlying vasculature in these burns may still be intact albeit with higher inflammatory blood perfusion. These are promising results as clinical LDI measurements from other groups have consistently shown that higher blood perfusion in superficial-partial thickness burns is well correlated with faster healing times, whereas lower perfusion in deep-partial and full thickness burns may warrant surgical treatment.

SFDI water concentration maps may be an indicator of edema formation. As seen in FIGS. 7 and 8, we were able to observe different increases in tissue water concentration for each type of burn presented. Superficial-partial thickness burns, though elevated, stayed about 10% above baseline during the entire post-burn period, whereas the deep-partial burns exhibited a large steady increase to about 45% above baseline over the course of the 3-h period. Burn injuries often cause extravasation of interstitial fluids into burn wounds and surrounding tissues. By nature of the collagen damage, these injuries often exhibit abnormal osmotic and hydrostatic pressure gradients that worsen with burn depth and normal inflammatory response. As suggested by Stamatas et al. in their hyperspectral imaging study of controlled skin inflammation, the interstitial fluid accumulation can eventually exert enough pressure upon both blood and lymphatic vessels leading to further proliferation of ischemia and edema buildup. It is possible that this mechanism may be responsible for the negative wound conversion found in many of our deep-partial thickness burn samples.

SFDI scattering b values may be an indicator of scattering moiety size due to tissue denaturation. As seen in FIGS. 9 and 10, changes in scattering b values varied depending on burn severity. Both superficial-partial and deep-partial thickness burns showed a gradual increase in b over the 3-h post-burn period, with the deep partial-thickness burn having the largest change in b. As described above, scattering b values are related to average optical scatter size present in the tissue, whereas higher b values are suggestive of smaller scattering radiuses and vice versa. Burn injuries often involve thermal denaturation of organelles, heterogeneous structures, and collagen (the “glassy” hyalinization observed in H&E histology). The results of this study suggest that these effects can manifest as smaller average scattering particle size compared to normal for superficial-partial thickness and deep-partial thickness burns. Apart from being statistically significant for the purpose of differentiating partial burn severities, b parameter maps were often the clearest in terms of spatially delineating the extent of the burned regions when compared to the SFDI maps for tissue oxygenation and water concentration.

The results of this study suggest that SFDI-derived data may be useful for early quantitative noninvasive assessment of burn wound severity. Here, we have demonstrated that SFDI can be used to visualize heterogeneous changes in blood oxygenation, water concentration, and optical scattering properties over a large (as opposed to microscopic) field-of-view, thereby allowing researchers and clinicians to better identify burn areas that are at risk of further vascular damage or edema progression. In addition to a longer term study to examine the combined predictive capability of these SFDI-related parameters in determining burn severity, use of the illustrated embodiments also include a multimodal studies utilizing both SFDI and a perfusion-based technique for understanding the complete vascular changes that occur within a burn wound.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed, in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A method using spatial frequency domain imaging (SFDI) for quantitative noninvasive, noncontact assessment of severity of burn injury to tissue in vivo comprising:
performing wide-field quantitative mapping of tissue optical properties including pixel-by-pixel demodulation and fitting of spatial frequency data performed with multispectral imaging to extract the local absorption and reduced scattering optical coefficients;
performing quantitative analysis of burn injury of tissue by separately analyzing absorption spectra at each pixel to yield a spatial map of blood oxygenation, water concentration, optical scattering changes or a combination thereof; and
classifying the severity of the burn injury to tissue according to the depth of the injury.

2. The method of claim 1 further comprising tracking wound progression of burn injury in tissue in vivo over time.

3. The method of claim 1 where performing quantitative analysis of burn injury of tissue by separately analyzing absorption spectra at each pixel to yield the spatial map of blood oxygenation comprises performing quantitative analysis of burn injury of tissue by separately analyzing absorption spectra at each pixel to the yield spatial map of local oxy-hemoglobin (ct0₂Hb), deoxy hemoglobin concentration (ctHHb), and water concentration (ctH₂0).

4. The method of claim 3 where performing quantitative analysis of burn injury of tissue by separately analyzing absorption spectra at each pixel to the yield spatial map of local oxy-hemoglobin (ct0₂Hb), deoxy hemoglobin concentration (ctHHb), and water concentration (ctH₂0) further comprises performing quantitative analysis of burn injury of tissue by separately analyzing absorption spectra at each pixel to yield the spatial map of total hemoglobin (ctHbT) and oxygen saturation (St0₂) calculated as ctHbT=ctHHb+ctHHb+ct0₂Hb and St0₂=100*ct0₂Hb/ctHbT, respectively.

5. The method of claim 4 further comprising tracking wound progression in burns using total hemoglobin (ctHbT) and oxygen saturation (St0₂).

6. The method of claim 1 where performing quantitative analysis of burn injury of tissue by separately analyzing absorption spectra at each pixel to the yield spatial map of optical scattering changes comprises recovering the reduced scattering parameters at discrete wavelengths on a pixel by pixel basis using SFDI and fitting the reduced scattering parameters to a power law (μₛ′=A*λ^{-b}) at each pixel, where A is a parameter proportional to the number of scattering particles in the turbid medium and b is a parameter proportional the average size of the scattering particles in the tissue.

7. The method of claim 6 further comprising measuring the wavelength dependent A and b parameters in a two dimensional spatial map to provide an indication of the thermal damage to the collagen fibril network, where the changes in A and b are caused by denaturation of collagen fibrils and can then be correlated to burn severity and extent.

8. The method of claim 1 further comprising assessing wound healing as characterized by changes in scattering that reflect the different phases of a healing process.

9. The method of claim 6 further comprising selecting regions of the spatial map to compare the A and b parameters for all selected regions where the A value and bare larger for selected regions with a more severe burn.

10. The method of claim 9 where the severity of the burn is assessed to be related to the amount of change in the A and b parameters.

11. The method of claim 6 where a change in A and b is due to a change in collagen fibril structures, where A is proportional to the number of scattering particles in the tissue and b is proportional the average size of the scattering particles in the tissue, when the tissue is burned, the collagen fibrils are denatured due to thermal interactions and the average scattering size gets smaller so that b increases, and the number of scattering particles will increase so that A increases, such that use of SFDI measures of the A and b parameters when displayed in a two dimensional map provide an indication of the thermal damage to the collagen fibril network with the changes in A and b being caused by denaturation of collagen fibrils correlated to burn severity and extent.

12. The method of claim 1 where performing quantitative analysis of burn injury of tissue by separately analyzing absorption spectra at each pixel to yield a spatial map of optical scattering changes further comprises using optical absorption data to map tissue chromophores for determining burn treatment.

13. The method of claim 1 performing quantitative analysis of burn injury of tissue by separately analyzing absorption spectra at each pixel to yield a spatial map of water concentration to predict buildup of edema and ischemia progression.

14. The method of claim 1 further comprising predicting burn injury healing progression based on SFDI-related parameters.

15. The method of claim 1 further comprising using multimodal imaging using SFDI and perfusion-based imaging to characterize and analyze vascular changes occurring within a burn wound.

16. A method using spatial frequency domain imaging (SFDI) for quantitative noninvasive, noncontact assessment of severity of burn injury to tissue in vivo comprising:
performing wide-field quantitative mapping of tissue optical properties;
separately analyzing optical properties to generate a spatial map of blood oxygenation, water concentration, optical scattering changes or a combination thereof; and
determining the severity of the burn injury to tissue according to the spatial map of blood oxygenation water concentration, optical scattering changes or a combination thereof.

17. The method of claim 16 further comprising tracking progression of burn injury in tissue in vivo over time to determine treatment of the burn injury.

18. The method of claim 16 further comprising treating the burn injury according to the spatial map of burn injury in terms of blood oxygenation, water concentration, optical scattering changes or a combination thereof.

19. The method of claim 16 where separately analyzing optical properties to generate a spatial map of blood oxygenation, water concentration, optical scattering changes or a combination thereof comprises separately analyzing absorption spectra at each pixel to generate the spatial map of local oxy-hemoglobin (ct0₂Hb), deoxy hemoglobin concentration (ctHHb), and water concentration (ctH₂0).

20. The method of claim 16 where separately analyzing optical properties to generate a spatial map of blood oxygenation, water concentration, optical scattering changes or a combination thereof comprises recovering the reduced scattering parameters at discrete wavelengths on a pixel by pixel basis using SFDI and fitting the reduced scattering parameters to a power law μₛ=A*λ^{-b}) at each pixel, where A is a parameter proportional to the number of scattering particles in the turbid medium and b is a parameter proportional the average size of the scattering particles in the tissue.
